(19) United States
(12) Patent Application Publication
(10) Pub. No.: US 2012/0203023 A1
(43) Pub. Date: Aug. 9, 2012

(54) Process For Preparing A Phenylalanine Derivative

(76) Inventors: Joseph Paul Adams, Stevenage (GB); Toru Kuroda, Osaka (JP); Hiroaki Matsumae, Osaka (JP); John Bryce Strachan, Stevenage (GB); Nobuhiro Yagi, Osaka (JP)

(21) Appl. No.: 13/500,148

(22) PCT Filed: Oct. 19, 2010
(86) PCT No.: PCT/EP10/65710

§ 371 Date: Apr. 4, 2012

Related U.S. Application Data
(60) Provisional application No. 61/253,638, filed on Oct. 21, 2009.

(51) Int. Cl. C07C 227/18; C07C 229/38

(52) U.S. Cl. 560/41; 562/444

(57) ABSTRACT

A novel process for the preparation of a phenylalanine derivative of formula (I):

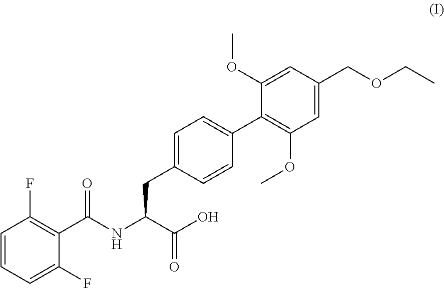

### TECHNICAL FIELD

The present invention relates to a novel process for the preparation of (2S)-2-{[(2,6-difluorophenyl)carbonyl]amino}-3-[4′-[(ethyloxy)methyl]-2′,6′-bis(methyloxy)-4-biphenylyl]propanoic acid and to intermediate products used therein.

### BACKGROUND TO THE INVENTION

International patent application WO 02/18320 (Tanabe Seiyaku Co., Ltd), filed 27 Aug. 2001, discloses novel phenylalanine derivatives that are inhibitors of α₄ (including α₄β₇ and α₄β₁) mediated adhesion. In particular, WO 02/18320 discloses (2S)-2-{[(2,6-difluorophenyl)carbonyl]amino}-3-[4′-[(ethyloxy)methyl]-2′,6′-bis(methyloxy)-4-biphenylyl]propanoic acid (referred to as Example 12—N-(2,6-difluorobenzoyl)-4-(2,6-dimethoxy-4-ethoxymethylphenyl)-L-phenylalanine) to which the novel process disclosed in this application relates. WO 02/18320 further discloses a process for the preparation of this compound of interest.

International patent application WO 03/072536 (Tanabe Seiyaku Co., Ltd), filed 27 Feb. 2003, outlines an alternative process for the preparation of phenylalanine derivatives, including the above-mentioned compound, (2S)-2-{[(2,6-difluorophenyl)carbonyl]amino}-3-[4′-[(ethyloxy)methyl]-2′,6′-bis(methyloxy)-4-biphenylyl]propanoic acid.

The object of the present invention is to provide an alternative process for the preparation of (2S)-2-{[(2,6-difluorophenyl)carbonyl]amino}-3-[4′-[(ethyloxy)methyl]-2′,6′-bis(methyloxy)-4-biphenylyl]propanoic acid (also known as N-(2,6-difluorobenzoyl)-4-(2,6-dimethoxy-4-ethoxymethylphenyl)-L-phenylalanine).

### SUMMARY OF THE INVENTION

The present invention provides a process for the preparation of the compound of formula (I):

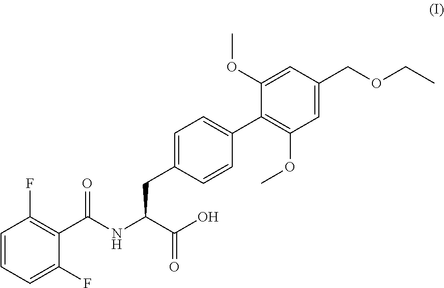

which process comprises the steps:

a) hydrolysis of an ester of formula (IIa):

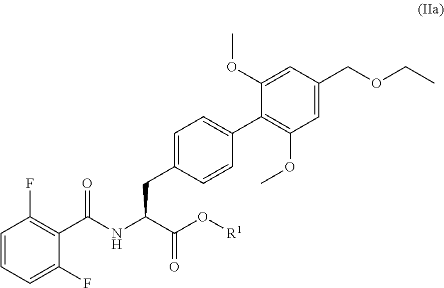

wherein R¹ is C₁₋₆ alkyl;

(b) formation of a solvate of the product obtained from step (a) (solvation);

(c) de-solvation of the solvate obtained from step (b) to yield the compound of formula (I); and

(d) optional re-crystallisation of the product obtained from step (c).

This alternative and simplified process for the preparation of the compound of formula (I) affords a pharmaceutical product with an improved impurity profile when compared to the compound of formula (I) prepared according to the prior art processes.

### BRIEF DESCRIPTION OF THE FIGURES

FIG. 1 shows the XRPD data for a crystalline form of the acetone solvate of the compound of formula (I).

FIG. 2a shows FT-IR data for a crystalline form of the acetone solvate of the compound of formula (I) (full spectral range 4000-675 cm⁻¹).

FIG. 2b shows FT-IR data for a crystalline form of the acetone solvate of a compound of formula (I) (fingerprint region 2000-675 cm⁻¹).

### DETAILED DESCRIPTION OF THE INVENTION

The present invention provides a process for the preparation of the compound of formula (I):

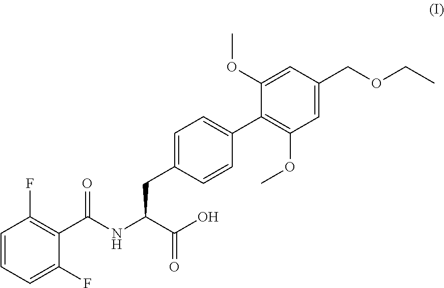

which process comprises the steps:

a) hydrolysis of an ester of formula (IIa):

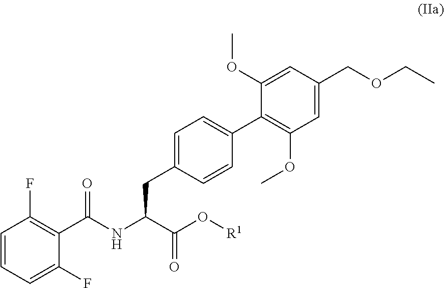

wherein R¹ is C₁₋₆ alkyl;

(b) formation of a solvate of the product obtained from step (a) (solvation);

(c) de-solvation of the solvate obtained from step (b) to yield the compound of formula (I); and

(d) optional re-crystallisation of the product obtained from step (c).

This alternative and simplified process for the preparation of the compound of formula (I) affords a pharmaceutical product with an improved impurity profile when compared to the compound of formula (I) prepared according to the prior art processes.

As used herein, the term “alkyl” refers to straight or branched hydrocarbon chains containing the specified number of carbon atoms. For example, C₁₋₆alkyl means a straight or branched alkyl chain containing at least 1, and at most 6, carbon atoms. Examples of “C₁₋₆alkyl” as used herein include, but are not limited to, methyl, ethyl, n-propyl and n-butyl, n-pentyl and n-hexyl.

In one aspect of the invention group R¹ is ethyl.

The ester hydrolysis of step (a) may be performed under acidic or basic conditions.

In one aspect of the invention the ester hydrolysis step is performed under basic conditions. Suitable bases include alkali metal hydroxides such as, but not limited to, potassium hydroxide, sodium hydroxide and lithium hydroxide. When the base employed is an alkali metal hydroxide, the ester hydrolysis proceeds via a carboxylate salt intermediate. This carboxylate salt intermediate may be isolated from the solvent. Thus in a further aspect of the invention, the ester hydrolysis of step (a) is performed under basic conditions employing an alkali metal hydroxide to afford the appropriate carboxylate salt, which may be isolated from the solvent. The appropriate carboxylate salt may exist in the form of a hydrate, such as a monohydrate or dihydrate. In yet a further aspect of the invention, the ester hydrolysis reaction is performed utilising potassium hydroxide, as a suitable base.

In a further aspect of the invention there is provided the potassium salt of the compound of formula (I):

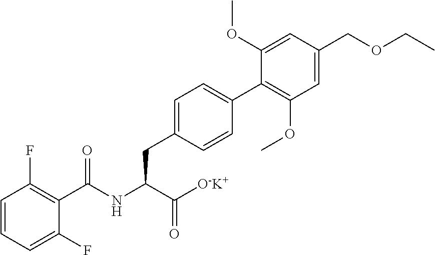

When the ester hydrolysis of step (a) is performed under basic conditions the reaction mixture is subjected to an acidic work up to afford the free acid. Appropriate acids for use in the acidic work up include inorganic acids, such as, but not limited to, hydrochloric acid and sulphuric acid, and organic acids with a pKa value lower than that of the compound of formula (I), such as, but not limited to, citric acid.

Suitable acids for effecting the ester hydrolysis of step (a) could include inorganic acids such as, but not limited to, hydrochloric acid, nitric acid, sulphuric acid, and organic acids such as, but not limited to, trifluoroacetic acid, p-toluenesulfonic acid.

The acidic or basic ester hydrolysis of step (a) may be performed in a suitable solvent, or mixture of solvents. Suitable solvents include water and organic solvents. Organic solvents include, but are not limited to, ethers (e.g., dioxane and tetrahydrofuran), acetonitrile and ketones (e.g., acetone and methyl ethyl ketone).

The acidic or basic ester hydrolysis of step (a) may be performed at room temperature or below.

Step (b), the formation of a solvate of the product of step (a) (solvation) may be achieved via the addition of the solvent from which the solvate will be derived to a solution of the product of step (a), followed by crystallisation and isolation of the product by filtration. Optionally, the crystallisation may be initiated by seeding with a crystal of the solvate.

In one aspect of the invention the product of step (a) may be solvated with a polar solvent which may be either protic or aprotic. In a further aspect of the invention solvation may be achieved employing a polar aprotic solvent as the solvate. In a further aspect of the invention the product of step (a) is solvated with a solvent selected from the group consisting of acetone, acetic acid, acetonitrile, nitromethane, dimethyl sulfoxide and dimethyl formamide. In another aspect of the invention the product of step (a) is solvated with acetone.

In a further aspect of the invention there is provided the acetone solvate of the compound of formula (I):

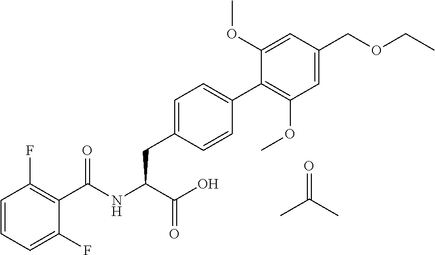

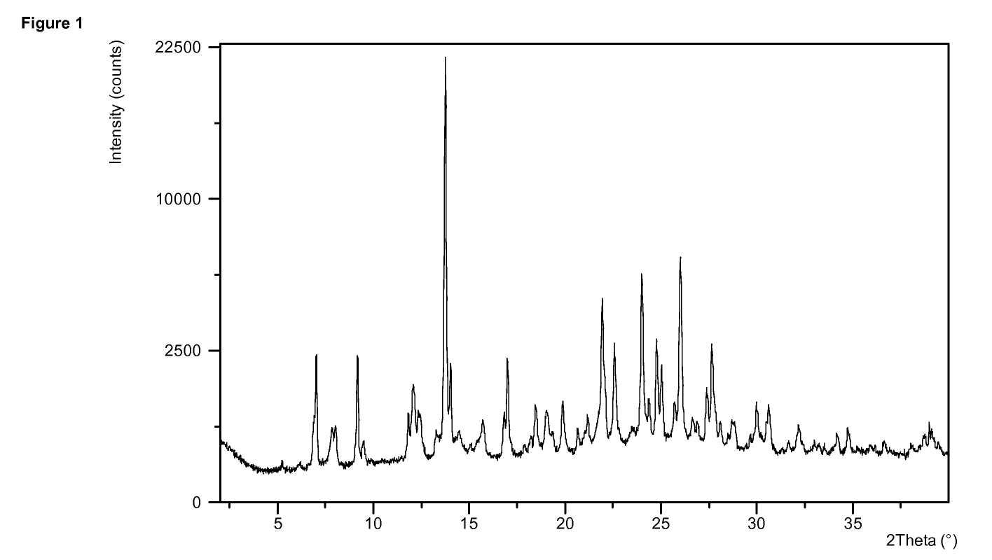

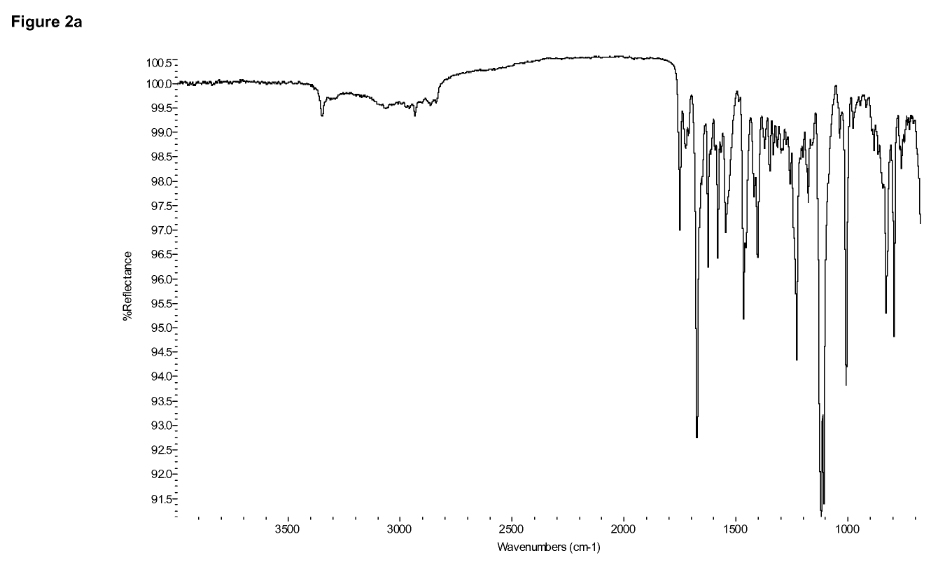

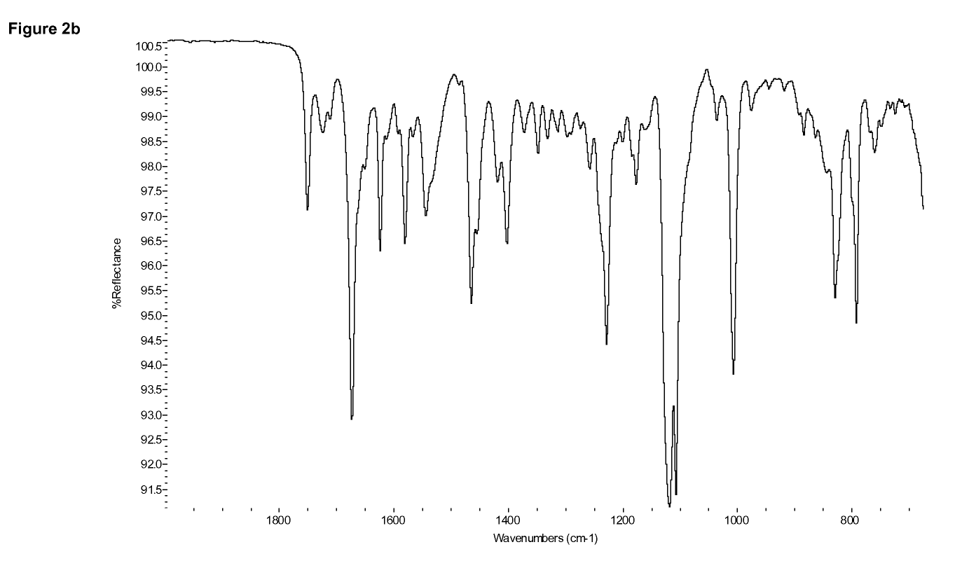

The acetone solvate of the compound of formula (I) may exist in crystalline form. Crystalline forms may be characterised by means of x-ray powder diffraction (XRPD) and/or by FT infra-red spectroscopy. Characterisation data for the crystalline acetone solvate of the compound of formula (I) are shown in FIGS. 1 and 2a/2b.

The invention provides for a crystalline form of the acetone solvate of the compound of formula (I) characterised by substantially the same X-ray powder diffraction (XRPD) pattern as shown in FIG. 1, wherein the XRPD pattern is expressed in terms of 2 theta angles and obtained with a diffractometer using copper Kα-radiation and/or substantially the same infra-red spectra as shown in FIGS. 2a and 2b.

XRPD data were acquired on a PANalytical X'Pert Pro powder diffractometer, equipped with an X'Celerator detector. The acquisition conditions were: radiation: Cu Kα, generator tension: 40 kV, generator current: 45 mA, start angle: 2.0° 2θ, end angle: 40.0° 2θ, step size: 0.0167° 2θ. The time per step was 31.750 s. The sample was prepared by mounting a few milligrams of sample on a silicon wafer (zero background) plates, resulting in a thin layer of powder.

Characteristic peak positions and calculated d-spacings are summarised in Table 1. These were calculated from the raw data using Highscore software. Experimental error in the peak positions is approximately ±0.1° 2θ. Relative peak intensities will vary due to preferred orientation.

TABLE 1
Characteristic XRPD peak positions for the acetone
solvate of the compound of formula (I).

| 2θ/° | d-spacing/Å |
|---|---|
| 6.9 | 12.9 |
| 7.0 | 12.6 |
| 9.2 | 9.6 |
| 11.8 | 7.5 |
| 12.1 | 7.3 |
| 13.7 | 6.4 |
| 14.0 | 6.3 |
| 17.0 | 5.2 |
| 18.5 | 4.8 |
| 19.9 | 4.5 |
| 21.9 | 4.1 |
| 22.6 | 3.9 |
| 24.0 | 3.7 |
| 24.8 | 3.6 |
| 26.0 | 3.4 |
| 27.6 | 3.2 |
| 30.0 | 3.0 |

The characteristic XPRD peaks of the crystalline acetone solvate of the compound of formula (I) are as follows: peaks at around 7.0, 9.2, 13.7, 14.0 and 24.0 degrees2 Theta.

The de-solvation step (step (c)) may be performed by heating the solvate of step (b) to yield the compound of formula (I). Alternatively, step (c) may be achieved by washing the solvate with a solvent capable of removing the solvate. Thus, in one aspect of the invention in step (c) the de-solvation is carried out either by drying or by washing the solvate of step (b).

In a further aspect the invention provides that the desolvation step (c) is performed by drying the solvate of step (b) under vacuum at a temperature between room temperature and the boiling point of the solvate.

Optionally, the compound of formula (I) obtained from step (c) may be further purified by re-crystallisation (step (d)). Re-crystallisation may be achieved using a range of standard techniques, such as cooling re-crystallisation or anti-solvent addition re-crystallisation. In cooling re-crystallisation the crystalline compound of formula (I) is dissolved in a suitable solvent at an elevated temperature, the solution is then slowly cooled and optionally seeded to afford crystals of the compound of formula (I) which may be isolated by filtration, washed using a suitable solvent, and then dried. In antisolvent addition re-crystallisation the crystalline compound of formula (I) is dissolved in a suitable solvent. Addition of an anti-solvent reduces the solubility of the compound in solution promoting the formation of crystals. Optionally, the solvent system may be seeded. The crystals of the compound of formula (I) thus formed may be isolated by filtration, washed using a suitable solvent and then dried.

In a further aspect of the invention the crystalline compound of formula (I) from step (c) may be dissolved in ethyl acetate at elevated temperature (for example at approximately 50° C.). The resulting solution may be treated with heptane, cooled and seeded with crystals of the compound of formula (I). The resulting crystals of the compound of formula (I) may then be isolated by filtration, washed and dried.

It will be appreciated by a person skilled in the art that certain steps in the chemical process described herein may be telescoped such that one or more intermediate products are not isolated before proceeding to the next stage in the process.

In a further aspect of the invention there is provided a process for the preparation of the compound of formula (I)

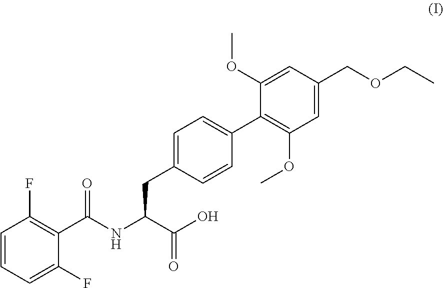

which process comprises the steps:

a) hydrolysis of the ester of formula (II):

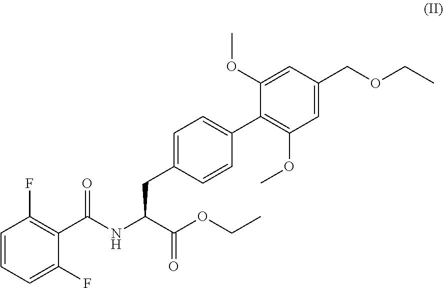

using potassium hydroxide, followed by an acidic work up employing citric acid;

(b) formation of an acetone solvate of the product obtained from step (a); and

(c) de-solvation of the acetone solvate obtained from step (b) via drying the solvate under vacuum at elevated temperature to yield the compound of formula (I).

In a further aspect of the invention the process for the preparation of the compound of formula (I) comprises the further step of re-crystallisation of the compound of formula (I) from ethyl acetate/heptane.

The compound of formula (II) may be prepared according to the methodology set out in Steps 1 and 2 of WO 03/072537 (Tanabe Seiyaku Co., Ltd). Alternatively, the compound of formula (II) may be prepared as described in WO 02/18320 (Tanabe Seiyaku Co., Ltd).

Compounds of formula (IIa) may also be prepared according to the reaction scheme set out below (Scheme 1):

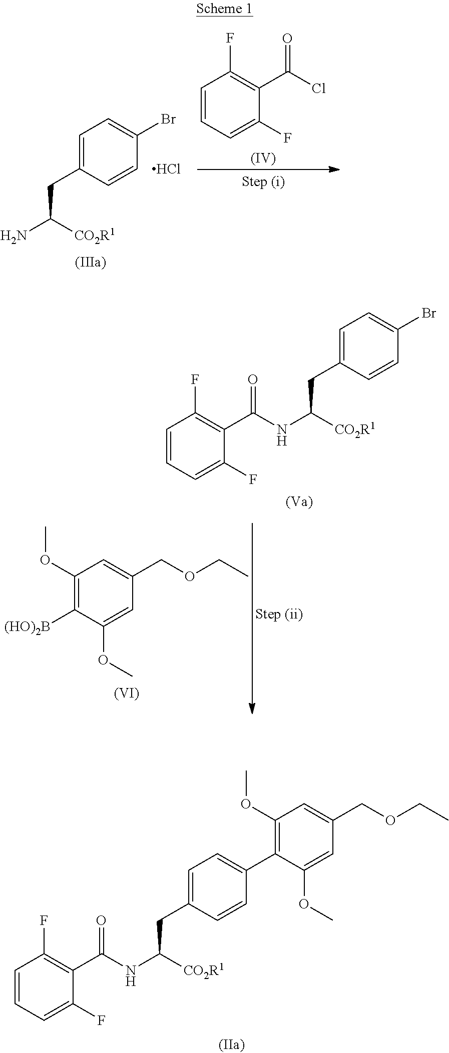

wherein R¹ is C₁₋₆alkyl.

Compounds of formula (Va) may conveniently be prepared under Step (i) above by reacting a compound of formula (IIIa) with a compound of formula (IV) in the presence of a suitable base (such as, but not limited to, potassium carbonate) in a suitable solvent (such as, but not limited to, MIBK) or mixture of solvents (such as, but not limited to, water and MeTHF).

Compounds of formula (IIa) may conveniently be prepared under Step (ii) above by coupling a compound of formula (Va) with a compound of formula (VI) under Suzuki coupling reaction conditions. Examples of suitable catalysts for use in a Suzuki coupling reaction include palladium catalysts, such as, but not limited to, palladium acetate, palladium chloride and dichlorobis(triphenylphosphine)palladium. In the case where the reaction is performed in the presence of a palladium (II) catalyst that does not have ligands, such as, but not limited to, palladium acetate or palladium chloride, it is necessary to add a phosphine (such as, but not limited to triphenylphosphine, tri-ortho-tolyl phosphine, tri-tert-butyl phosphine or di-phenyl cyclo-hexyl phosphine) or a phosphite (such as, but not limited to, triethylphosphite) in order to facilitate the reaction. Examples of suitable bases that can be used in the Suzuki coupling reaction include inorganic bases, such as, but not limited to, alkali metal carbonates, and organic bases, such as, but not limited to, alkylamines (diisopropylamine, triethylamine and diisopropylethylamine). The Suzuki coupling reaction under Step (ii) shall be performed in a suitable solvent or mixture of solvents (such as, but not limited to, water and MeTHF).

The compound of formula (II) may be prepared according to the reaction scheme set out below (Scheme 2):

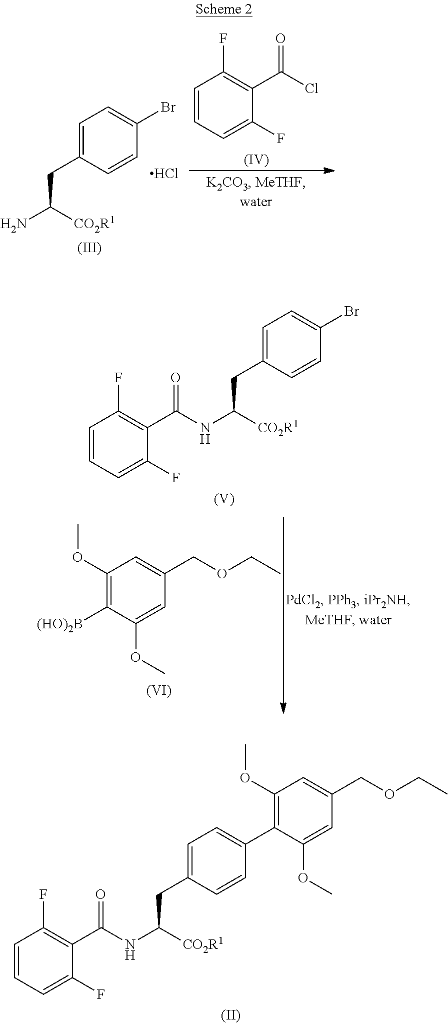

wherein R¹ is ethyl.

In a further aspect the present invention provides for a process for the preparation of a compound of formula (II) which comprises coupling the compound of formula (V)

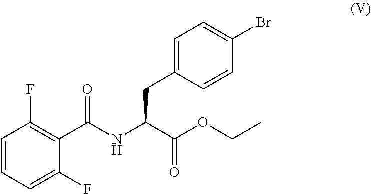

with the compound of formula (VI)

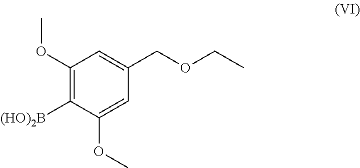

Suitable coupling conditions for the compound of formula (V) and the compound of formula (VI) include those shown in Scheme 2.

In a further aspect of the invention there is provided the compound of formula (V):

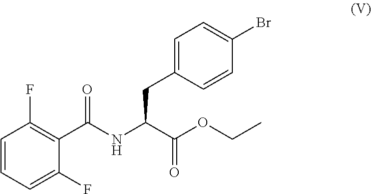

¹H NMR characterisation data for the compound of formula (V) were generated on an isolated and purified batch. ¹H-NMR spectra were recorded on a Bruker Avance 400 at 400 MHz, using TMS as an internal reference.

¹H NMR (400 MHz, DMSO-D6) δ ppm 1.17 (t, J=7.09 Hz, 3H) 2.96 (dd, J=13.82, 9.90 Hz, 1H) 3.11 (dd, J=13.82, 5.26 Hz, 1H) 4.12 (q, J=7.09 Hz, 2H) 4.63 (ddd, J=9.78, 7.82, 5.38 Hz, 1H) 7.15 (t, J=7.95 Hz, 2H) 7.25 (d, J=8.31 Hz, 2H) 7.47-7.55 (m, 3H) 9.23 (d, J=7.83 Hz, 1H).

Experimental

Analytical Equipment

¹H-NMR spectra were recorded on a Bruker Avance 400 at 400 MHz, using TMS as an internal reference.

Infrared absorption spectrums were recorded over the wavenumber range 4000 to 650 cm⁻¹ using a Perkin Elmer Spectrum One FT-IR spectrometer equipped with a Perkin Elmer Universal ATR (attenuated total reflection) sampling accessory.

### SPECIFIC EXAMPLE

The invention is illustrated in the following non limiting example.

### Preparation of ethyl(2S)-2-{[(2,6-difluorophenyl)carbonyl]amino}-3-[4′-[(ethyloxy)methyl]-2′,6′-bis(methyloxy)-4-biphenylyl]propanoate

To a solution of potassium carbonate (18.8 Kg, 136.04 mol) in water (70 L) is added ethyl-4-bromo-L-phenylalaninate hydrochloride (14 Kg, 45.37 mol, supplied by DowPharm) and Me-THF (70 L). The biphasic mixture is cooled to 10±3° C. and 2,6-difluorobenzoyl chloride (8.4 Kg, 47.58 mol, supplied by Shanghai Chemspec) is added keeping the temperature below 15° C. The reaction is then stirred for 30 minutes while warming up to 25±3° C. The phases are then separated. To the organic phase, containing ethyl 4-bromo-N-[(2,6-difluorophenyl)carbonyl]-L-phenylalaninate, 4-[(ethyloxy)methyl]-2,6-bis(methyloxy)phenyl]boronic acid (11.4 Kg, 47.5 mol, supplied by Juzen) is added. The organic phase is then diluted with Me-THF (28 L) and water (18.2 L). Palladium chloride (23.8 g, 0.13 mol) and triphenylphosphine (71.4 g, 0.27 mol) are added and the vessel is purged three times with nitrogen to remove all traces of air. Diisopropylamine (9.5 L, 67.93 mol) is added and the purge is repeated. The reaction mixture is then heated to 75±3° C. (reflux) for about 3 hours. Once complete by HPLC the solution is cooled to 60±3° C. and L-cysteine (2.8 Kg) is added. The reaction mixture is heated at 60±3° C. for 2 hours. After this time the reaction mixture is cooled to 25±3° C. 2M hydrochloric acid (28 L) is added. After stirring for 10 min the layers are separated. The organic phase is then washed with saturated aqueous sodium bicarbonate (28 L). The layers are again separated and the organic layer passed through a Domnic Hunter filter cartridge, washing with Me-THF (7 L). The organic phase is then concentrated to 28 L via atmospheric distillation. Isopropyl alcohol (84 L) is added and the solution is concentrated to 28 L. Isopropyl alcohol (84 L) is again added and the solution is concentrated to 84 L. A sample is taken to ensure Me-THF levels are <0.2 eq. Heptane (95%) (84 L) is added maintaining the contents above 55° C. and the solution is cooled to 45±3° C. before a seed of ethyl(2S)-2-{[(2,6-difluorophenyl)carbonyl]amino}-3-[4′-[(ethyloxy)methyl]-2′,6′-bis(methyloxy)-4-biphenylyl]propanoate (70 g) is added and the slurry aged for about 30 minutes. The thin slurry is cooled to 38° C. and held for 1 hour. It is then re-heated to 45° C. and held for 45 minutes. The resulting slurry is cooled to 10° C. over 2 hours and held for 1 hour. The solid is then collected by filtration and washed with isopropyl alcohol:heptane (95%) (1:4, 2×28 L). The product is then dried in vacuo at 50° C. to give the product (20.35 Kg, 85%).

¹H NMR (400 MHz, DMSO-D6) δ ppm 1.17 (dt, J=16.08, 7.00 Hz, 6H) 3.08 (ddd, J=19.81, 14.06, 5.50 Hz, 2H) 3.53 (q, J=7.01 Hz, 2H) 3.65 (s, 6H) 4.04-4.16 (m, 2H) 4.47 (s, 2H) 4.60-4.68 (m, 1H) 6.69 (s, 2H) 7.09-7.18 (m, 4H) 7.24 (d, J=8.07 Hz, 2H) 7.51 (ddd, J=14.92, 8.31, 6.60 Hz, 1H) 9.31 (d, J=7.58 Hz, 1H)

### Preparation of (2S)-2-{[(2,6-difluorophenyl)carbonyl]amino}-3-[4′-[(ethyloxy)methyl]-2′,6′-bis(methyloxy)-4-biphenylyl]propanoic acid

Ethyl(2S)-2-{[(2,6-difluorophenyl)carbonyl]amino}-3-[4′-[(ethyloxy)methyl]-2′,6′-bis(methyloxy)-4-biphenylyl]propanoate (15 Kg) was taken up in tetrahydrofuran (37.5 L) and passed through a CUNO filter containing charcoal (R55SP). Tetrahydrofuran (37.5 L) and water (45 L) were added and the resulting mixture cooled to 10±3° C. Aqueous KOH (4.65 Kg, 45% w/w) was added and the mixture stirred at 10±3° C. until the reaction was complete. Aqueous citric acid (18.15 Kg, 50% w/v) was charged followed by toluene (75 L). The reaction mixture was heated to 50±3° C. and the aqueous phase discharged to waste. The organic phase was washed with water (2×30 L) at 50±3° C. The organic phase was then concentrated to 75 L by atmospheric distillation. Toluene (45 L) and acetone (75 L) were charged and the solution concentrated to 120 L. Acetone (75 L) was again charged and the solution again concentrated to 105 L. Toluene (75 L) was charged, keeping T>55±3° C. The solution was cooled to 35° C., seeded with (2S)-2-{[(2,6-difluorophenyl)carbonyl]amino}-3-[4′-[(ethyloxy)methyl]-2′,6′-bis(methyloxy)-4-biphenylyl]propanoic acid (acetone solvate) (75 g) and cooled to 0±3° C. over 4 hrs and held at this temp for 1 hr. The solid product was isolated by filtration, washing with cold (<5° C.) toluene/acetone (45 L, 10:1), cold (<5° C.) toluene (45 L) and dried in vacuo at 70° C. to give the product (10.1 Kg, 71%).

### Recrystallisation of (2S)-2-{[(2,6-difluorophenyl)carbonyl]amino}-3-[4′-[(ethyloxy)methyl]-2′,6′-bis(methyloxy)-4-biphenylyl]propanoic acid

(2S)-2-{[(2,6-difluorophenyl)carbonyl]amino}-3-[4′-[(ethyloxy)methyl]-2′,6′-bis(methyloxy)-4-biphenylyl]propanoic acid (9.38 Kg) was charged into a clean reactor, followed by ethyl acetate (46.9 L). The solution was heated to 50° C. and filtered into the pre-warmed (35° C.) crystallizing vessel. A line-wash with ethyl acetate (9.4 L) was carried out. The combined ethyl acetate solutions were heated to 50° C., stirred to ensure complete dissolution. Filtered heptane (9.4 L) was added maintaining the temperature at 50° C. then the solution cooled to 30° C. and seeded with (2S)-2-{[(2,6-difluorophenyl)carbonyl]amino}-3-[4′-[(ethyloxy)methyl]-2′,6′-bis(methyloxy)-4-biphenylyl]propanoic acid (47 g) slurried in 1:9 ethyl acetate:heptane (0.47 L). The slurry was aged for 2 hours at 30° C. Filtered heptane (75 L) was added over 3 hours. The slurry was then cooled to 0° C. over 1 hour. The mixture was aged at 0° C. for 1 hour then the solid was filtered off, washed with isopropyl ether (29.6 L and dried under vacuum at 50±3° C. to give the product (8.55 Kg, 91%).

Characterised by having an infrared absorption spectrum with significant absorption bands at about 754, 768, 800, 820, 849, 866, 1006, 1100, 1122, 1157, 1188, 1225, 1242, 1268, 1292, 1317, 1352, 1417, 1466, 1530, 1580, 1624, 1650, 1662, 1711, 1728, 2938, 3302 cm⁻¹

What is claimed is:

1. A process for the preparation of the compound of formula (I): 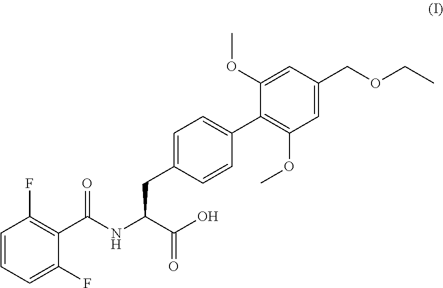
which process comprises the steps:
a) hydrolysis of an ester of formula (IIa): 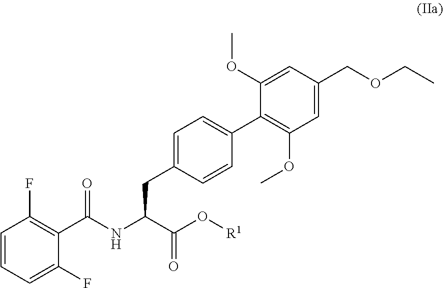
wherein R¹ is C₁₋₆ alkyl;
(b) formation of a solvate of the product obtained from step (a) (solvation);
(c) de-solvation of the solvate obtained from step (b) to yield the compound of formula (I); and
(d) optional re-crystallisation of the product obtained from step (c).

2. A process according to claim 1 in which R¹ is ethyl.

3. A process according to claim 1, wherein the ester hydrolysis step (step (a)) is performed under basic conditions.

4. A process according to claim 3, wherein the ester hydrolysis of step (a) is performed under basic conditions employing an alkali metal hydroxide to afford the appropriate carboxylate salt, which may be isolated from the solvent.

5. A process according to claim 4, wherein the ester hydrolysis of step (a) is performed utilising potassium hydroxide.

6. A process according to claim 1, wherein in step (b), the product of step (a) may be solvated with a polar solvent which may be either protic or aprotic.

7. A process according to claim 6, wherein in step (b) the product of step (a) is solvated with a solvent selected from the group consisting of acetone, acetic acid, acetonitrile, nitromethane, dimethyl sulfoxide and dimethylformamide.

8. A process according to claim 7 wherein the product of step (a) is solvated with acetone.

9. A process according to claim 1, wherein in step (c) the de-solvation is carried out either by drying or by washing the solvate of step (b).

10. A process according to claim 9, wherein the desolvation step (c) is performed by drying the solvate of step (b) under vacuum at a temperature between room temperature and the boiling point of the solvate.

11. A process for the preparation of the compound of formula (I) 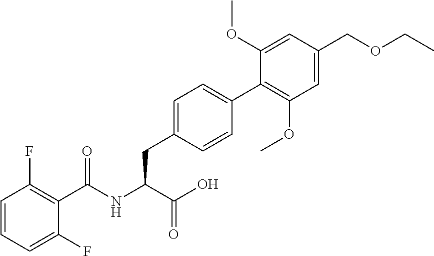
which process comprises the steps:
a) hydrolysis of the ester of formula (II): 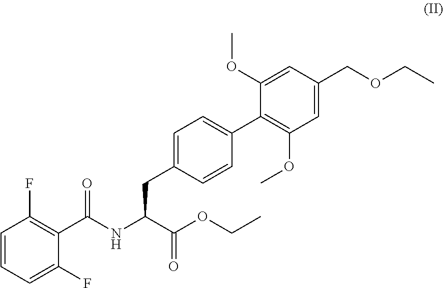
using potassium hydroxide, followed by an acidic work up employing citric acid;
(b) formation of an acetone solvate of the product obtained from step (a); and
(c) de-solvation of the acetone solvate obtained from step (b) via drying the solvate under vacuum at elevated temperature to yield the compound of formula (I).

12. The potassium salt of the compound of formula (I):

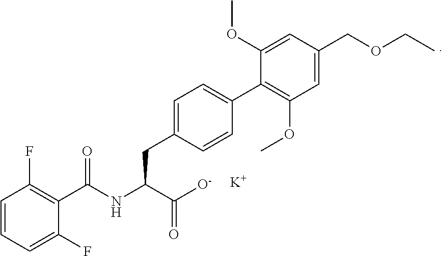

13. The acetone solvate of the compound of formula (I):

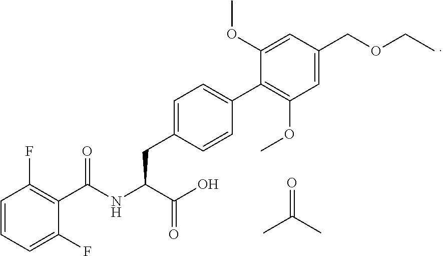

14. A crystalline form of the acetone solvate of the compound of formula (I) 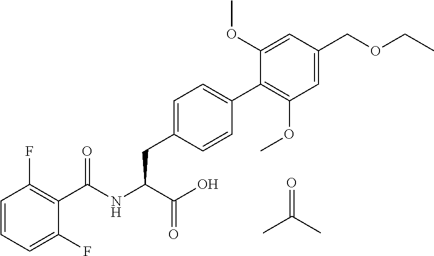
characterised by substantially the same X-ray powder diffraction (XRPD) pattern as shown in FIG. 1, wherein the XRPD pattern is expressed in terms of 2 theta angles and obtained with a diffractometer using copper Kα-radiation and/or substantially the same infra-red spectra as shown in FIGS. 2a and 2b.

15. A crystalline form according to claim 14 having characteristic XPRD peaks at around 7.0, 9.2, 13.7, 14.0 and 24.0 degrees2 Theta.

16. A process according to claim 11 in which the compound of formula (II) is prepared by coupling the compound of formula (V) 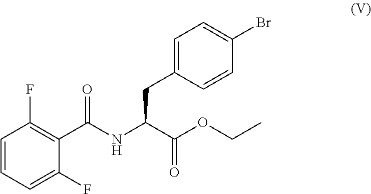
with the compound of formula (VI)

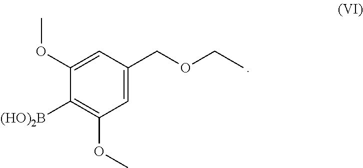

17. A compound of formula (V): [chemical structure]
